SEQUENCE LISTING (4 sequences)

SEQ ID NO: 1 - DNA, 20 bp, Homo sapiens
  source 1..20: mol_type = other DNA
cctttggctttgctgcactc

SEQ ID NO: 2 - DNA, 21 bp, Homo sapiens
  source 1..21: mol_type = other DNA
acggggttgttgtaagggtca

SEQ ID NO: 3 - DNA, 22 bp, Homo sapiens
  source 1..22: mol_type = other DNA
gtctcctctgacttcaacagcg

SEQ ID NO: 4 - DNA, 22 bp, Homo sapiens
  source 1..22: mol_type = other DNA
accaccctgttgctgtagccaa

(19) United States
(12) Patent Application Publication
(10) Pub. No.: US 2025/0114286 A1
(43) Pub. Date: Apr. 10, 2025

(54) METHOD FOR STABILIZING CERAMIDE AND COSMETIC COMPOSITION COMPRISING STABILIZED CERAMIDE

(71) Applicants: COSMECCA KOREA Co., Ltd., Chungcheongbuk-do (KR); ABIO MATERIALS Co., Ltd., Gyeonggi-do (KR)

(72) Inventors: Hang Eui CHO, Gyeonggi-do (KR); Jae Yong SEO, Chungcheongbuk-do (KR); Eun Ji KIM, Gyeonggi-do (KR); Hwi Yeob KIM, Gyeonggi-do (KR); Ji Soo RYU, Gyeonggi-do (KR); Jong Won JEON, Incheon (KR); Jung Soo KIM, Gyeonggi-do (KR); Eun Jeong YOON, Gyeonggi-do (KR); Min Ha KIM, Gyeonggi-do (KR); Jin Hong KIM, Gyeonggi-do (KR); So Hyeon BAE, Chungcheongnam-do (KR); Si Jun PARK, Gyeonggi-do (KR); Hyun Sang LEE, Gyeonggi-do (KR)

(21) Appl. No.: 18/911,278

(22) Filed: Oct. 10, 2024

(30) Foreign Application Priority Data
Oct. 10, 2023 (KR) 10-2023-0134339

(51) Int. Cl. A61K 8/42; A61K 8/34; A61K 8/63; A61Q 19/00

(52) U.S. Cl. CPC A61K 8/42; A61K 8/347; A61K 8/63; A61Q 19/005; A61Q 19/007

(57) ABSTRACT

The present invention relates to a method for stabilizing ceramide and to a cosmetic composition containing stabilized ceramide and, more specifically, to a method for homogenizing and stabilizing ceramide by self-assembling the ceramide together with β-sitosterol, resveratrol, and allantoin which are other physiologically active substances using a microfluidic chip and a high pressure homogenizer, and to a technique for using ceramide and nanoparticles containing β-sitosterol, resveratrol, and allantoin, which are produced by means of the stabilizing method, as a cosmetic material for skin moisturizing, anti-oxidation, skin inflammation relief, skin barrier enhancement, and skin itching relief.

### CROSS-REFERENCE TO RELATED APPLICATION

This application claims priority to and the benefit of Korean Patent Application No. 10-2023-0134339, filed Oct. 10, 2023, the disclosure of which is incorporated herein by reference in its entirety.

### SEQUENCE LISTING

The present application contains a Sequence Listing which has been submitted electronically in XML format and is hereby incorporated by reference in its entirety. The Sequence Listing, created on Nov. 25, 2024, is 4,587 bytes in size and is named NP_PFP240010US_Sequence Listing.XML.

### BACKGROUND

### Technical Field

The present invention relates to a method for stabilizing ceramide and a cosmetic composition containing stabilized ceramide and, more specifically, to a technique for stabilizing ceramide by preparing ceramide as nanoparticles together with β-sitosterol, resveratrol, and allantoin, which are bioactive substances, by applying a low-temperature microfluidic chip technology, and using the ceramide as a cosmetic material.

### Background Art

Cosmetics have been used for the purpose of protecting the skin and imparting beauty. However, in recent years, functional cosmetics with various functions such as skin wrinkles, elasticity improvement, anti-oxidation, and whitening are in the spotlight beyond the existing simple skin protection or moisturizing by emphasizing functionality. Accordingly, ingredients with various activities are contained, which may cause skin problems or inflammation.

In addition, in the case of cosmetics containing such various active ingredients, it is difficult to maintain formulation stability and the skin absorption rate is low, so it is difficult to expect sufficient skin improvement activity as desired when using the cosmetics.

Among these active ingredients, ceramide is one of sphingolipids having a structure in which a fatty acid is linked to sphingosine or phytosphingosine. Ceramide accounts for about 40% or more of the lipids between keratinocytes constituting the stratum corneum of skin, and is a component of the cell membrane and the skin lipid membrane of a double layer. In addition, ceramides are essential components to show the structure formation or function of the stratum corneum, and ceramides present in the human body are classified into various types according to the degree of polarity. Ceramide is an important element of the skin barrier, and has a function of inhibiting the evaporation of moisture and a function of maintaining the ordered structure of the stratum corneum, and thus, it is attracting attention as a raw material for moisturizing cosmetics. Such ceramide is known to decrease in amount as skin aging progresses, and accordingly, the function of the skin barrier decreases, and thus, a decrease in the amount of moisture in the skin, an increase in the loss of moisture, an exfoliation phenomenon of the stratum corneum, and the like occur, thereby becoming a damaged or unhealthy skin barrier. Such symptoms can be improved by supplying ceramide to the skin, but ceramide is a poorly soluble material, and due to its structural specificity, it is limited to contain a large amount of ceramide in the cosmetic material, and since it is not easy to solubility, it is difficult to secure usability, transparency, and formulation stability of the cosmetics, and thus there is a limit to its utilization.

The present inventors studied stabilization of ceramide in formulation and improvement of skin absorption rate, and completed the present invention by confirming that ceramide, an active ingredient for improving skin barrier and pruritus, is prepared into nanoparticles by applying microfluid chip technology together with other physiologically active substances, β-sitosterol, resveratrol and allantoin, stability of the active ingredients is improved, skin absorption rate is improved, and excellent skin improvement activity is exhibited due to the synergistic effect thereof.

### SUMMARY

### Technical Problem

The present invention is to provide a method for stabilizing ceramide.

In addition, another object of the present invention is to provide a cosmetic composition which contains stabilized ceramide and thus exhibits excellent skin moisturizing, anti-oxidation, anti-inflammation, skin soothing, skin barrier enhancement, and skin irritation relief effects.

### Technical Solution

According to the present invention to achieve the above object, there is provided a method for stabilizing ceramide, comprising the steps of: (A) preparing a freeze-dried powder containing ceramide, resveratrol, and β-sitosterol; (B) b1) dissolving the freeze-dried powder in dipropylene glycol, and then mixing an emulsion polymer to prepare an oil phase portion; (b2) mixing purified water, dipropylene glycol, a water-soluble solubilizer, and allantoin to prepare an aqueous phase portion; (C) passing the oil phase portion and the aqueous phase portion through a microfluidic chip to self-assemble the oil phase portion and the aqueous phase portion; and (D) forming nanoparticles using a microfluidizer.

Preferably, the step (A) comprises the steps of: a1) adding ceramide, resveratrol, and β-sitosterol to warm dipropylene glycol and ethanol, followed by mixing, and dissolving; a2) subjecting the mixture to stand at room temperature followed by filtration to remove a precipitate; and a3) concentrating the mixture at 40 to 50° C., followed by freeze-drying the concentrate, thereby preparing a freeze-dried powder containing ceramide, resveratrol, and β-sitosterol.

The emulsion polymer may be at least one selected from the group consisting of phosphatidyl choline, Poly-ε-caprolactone (PCL), polyglycolic acid (PGA), poly-L-lactide (PLLA), sphingomyelin, phosphatidyl serine, phosphatidyl inositol, phosphatidyl ethanolamine, and lecithin.

The ceramide is at least one selected from the group consisting of ceramide NP, ceramide EOP, ceramide NS, ceramide AS, ceramide EOS, and ceramide NDS.

Preferably, in step a1), the ceramide, resveratrol and β-sitosterol are added and mixed at a weight ratio of 10:0.5 to 1.5:0.005 to 0.02, respectively.

Preferably, in the step b1), the oil-phase portion is formed by mixing 0.5 to 1% by weight of the freeze-dried powder, 80 to 85% by weight of dipropylene glycol, and 14 to 18% by weight of the emulsion polymer.

Preferably, in step b2), the aqueous phase part is composed of 88 to 90% by weight of purified water, 8 to 10% by weight of dipropylene glycol, 1 to 2% by weight of a water-soluble solubilizer, and 0.0005 to 0.002% by weight of allantoin.

Preferably, step (C) comprises preparing the liposome which is self-assembled by passing the microfluidic chip, in which a flow channel of 1 um to 3 um is formed, 1 to 5 times under a condition of 10 to 15° C. by using the oil phase part and the water phase part at a weight ratio of 1:7 to 12, respectively.

In step (D), the nanoparticles are characterized in that they have a size of 50 to 300 nm.

In order to achieve the other objects, according to the present invention, there is provided a cosmetic composition containing 0.0001 to 50.0 wt % of ceramide stabilized by the method based on the total weight of the composition.

The cosmetic composition is used for skin moisturizing, anti-oxidation, skin inflammation relief, strengthening skin barrier enhancement or skin itching relief.

The cosmetic composition has a formulation such as skin lotion, skin toner, pack, nourishing cream, moisture cream, essence, body cream, body lotion, body oil, shampoo, rinse, hair conditioner, hair gel, cleansing foam, cleansing lotion, soap, patch, foundation, lipstick, makeup base, etc.

### BRIEF DESCRIPTION OF THE DRAWINGS

FIG. 1 is a graph showing the results of particle size analysis of nanoparticles prepared in Examples of the present invention.

### DETAILED DESCRIPTION

Hereinafter, the present invention will be described in more detail.

The present invention, in stabilizing ceramide as an active ingredient with liposome, improves stability of the active ingredient and skin absorption rate by freeze-drying and homogenizing with resveratrol, β-sitosterol, and allantoin using a low temperature microfluidic chip and a high pressure homogenizer.

The nanoparticle capsule, which comprises ceramide and other physiologically active substances such as resveratrol, β-sitosterol, and allantoin, is formed as a structure of a liposome dual layer, and has high skin absorption rate, stays in the skin for a long time, stably absorbs the active substance, and thus has an advantage of efficiently securing an effect of improving the skin.

Resveratrol exhibits various physiological and pharmacological actions, such as strong antibacterial activity, antiviral activity, anti-oxidation efficacy, antithrombotic activity, and anti-inflammatory activity. Accordingly, it has been confirmed to be helpful for cardiovascular protective effect, cancer prevention, and anticancer activity, and its potential as a cancer prevention agent has been proven, and it is also in the spotlight as a component of food or cosmetics. However, resveratrol is a poorly soluble ingredient that is extremely limitedly used in water-soluble processing of beverages and the like, and trans-type resveratrol is changed into cis-type having low bioactivity when exposed to light, heat, oxygen, and the like due to structural instability, and metabolism is fast and a half-life thereof is short in the body, and thus bioavailability is very low when ingested. Therefore, in order to expand and develop the resveratrol industry, there is an urgent need to develop a technology capable of increasing the bioavailability and stability of resveratrol.

β-sitosterol is a fat-soluble material having a chemical structure similar to cholesterol in the human body, and is a main component of Insadol™, which is a treatment for periodontal diseases due to anti-inflammatory, antibacterial, and anti-oxidation effects. However, β-sitosterol is a fat-soluble material, and is not mixed with a general aqueous pharmaceutical composition, but separated and aggregated to form a lump, and when the composition is applied to the skin, it does not spread over the entire skin but aggregates, and thus it is difficult to uniformly apply the composition to the skin and is not stable, and thus there is a problem in formulation development.

Allantoin increases the function of cell interstitium to remove keratin, moisturizes the skin to soften the skin, and promotes the germination and reproduction of cells to help heal wounds on the skin. In addition, allantoin may be used as a component of drugs or cosmetics that dissolve dead skin cells to help the drug penetrate well, and supplies moisture to the wound tissue to help heal the wound tissue, thereby promoting metabolism.

According to the present invention, a method for stabilizing ceramide comprises the steps of: (A) preparing a freeze-dried powder containing ceramide, resveratrol, and β-sitosterol; (B) b1) dissolving the freeze-dried powder in dipropylene glycol, and then mixing an emulsion polymer so as to prepare an oil phase; (b2) mixing purified water, dipropylene glycol, a water-soluble solubilizer, and allantoin so as to prepare an water phase; (C) allowing the oil phase and the water phase to pass through a microfluidic chip so as to self-assemble the same; and (D) forming nanoparticles using a microfluidizer.

Preferably, the step (A) comprises the steps of: a1) adding ceramide, resveratrol, and β-sitosterol to warm dipropylene glycol and ethanol, followed by mixing, and dissolving; a2) subjecting the mixture to stand at room temperature followed by filtration to remove a precipitate; and a3) concentrating the mixture at 40 to 50° C., followed by freeze-drying the concentrate, thereby preparing a freeze-dried powder containing ceramide, resveratrol, and β-sitosterol.

According to one embodiment of the present invention, ceramide, resveratrol and β-sitosterol are added to a mixed solvent of dipropylene glycol and ethanol heated to a temperature of 45° C. or higher, preferably 45 to 60° C., and the mixed solvent is dissolved.

The ceramide is at least one selected from the group consisting of ceramide NP, ceramide EOP, ceramide NS, ceramide AS, ceramide EOS, and ceramide NDS. In this case, the ceramide, resveratrol and β-sitosterol are added and mixed at a weight ratio of 10:0.5 to 1.5:0.005 to 0.02, respectively.

The solution is allowed to stand at room temperature at 15 to 25° C. for 20 to 30 hours, filtered to remove precipitates, and centrifuged to recover the supernatant. In order to obtain a high-purity powder after concentrating the supernatant at 40 to 50° C., the concentrate is rapidly frozen at −60° C. or less and freeze-dried to prepare a freeze-dried powder containing ceramide, resveratrol, and β-sitosterol.

Subsequently, the freeze-dried powder is dissolved in dipropylene glycol, and then an emulsion polymer is mixed to prepare an oil phase.

As the emulsion polymer, at least one selected from the group consisting of phosphatidyl choline, Poly-ε-caprolactone (PCL), polyglycolic acid (PGA), Poly-L-lactide (PLLA), Sphingomyelin, phosphatidyl serine, phosphatidyl inositol, phosphatidyl ethanolamine, and lecithin may be used.

According to one embodiment of the present invention, the oil phase is composed of 0.5 to 1 wt % of the freeze-dried powder, 80 to 85 wt % of the dipropylene glycol, and 14 to 18 wt % of the emulsion polymer.

Subsequently, purified water, dipropylene glycol, a water-soluble solubilizer and allantoin are mixed to prepare a water phase. According to one embodiment of the present invention, the water phase is composed of 88 to 90 wt % of purified water, 8 to 10 wt % of dipropylene glycol, 1 to 2 wt % of a water-soluble solubilizer, and 0.0005 to 0.002 wt % of allantoin. Polyglyceryl-10 Laurate may be used as the water-soluble solubilizer.

Subsequently, the prepared water phase and the oil phase are mixed and emulsified to stabilize. In the present invention, a microfluidic chip and a high pressure homogenizer are used in the preparation of an effective stabilizing formulation.

A microfluidic chip is a chip configured to flow a fluid through a microchannel based on microfluidics (microfluidic engineering) technology to perform various experiments and diagnosis. Microfluidic chip technology has been applied to emulsion preparation. Korean Patent Application Publication No. 10-2015-0126561 discloses a microfluidic chip and a method for preparing liposomes using the same, in which two types of solutions are injected after interposing fine thin plates between upper and lower plates to be laminated, and the flow rate ratio of each solution is adjusted to generate liposomes with adjusted uniformity and particle size.

According to one embodiment of the present invention, the oil phase and water phase are used in a ratio of 1:7 to 1:12 and passed through a microfluidic chip in which a microinjection port of 1 um to 3 um is formed, at a temperature of 15° C. or lower, preferably 10 to 15° C., at least once, and preferably 1 to 5 times, to prepare self-assembled liposomes.

Then, for secondary stabilization, the liposomes are formed into nanoparticles using a microfluidizer to stabilize ceramide.

According to an embodiment of the present invention, ceramide is stabilized by forming nanoparticles having a size of 50 to 300 nm under a condition of 300 to 500 bar.

The stability of ceramide is further improved by the addition of resveratrol, β-sitosterol, and allantoin, which are bioactive substances, when nanoliposome particles are prepared, and thus the stability and skin permeability of resveratrol, β-sitosterol, and allantoin are also improved. In addition, ceramide, β-sitosterol, resveratrol, and allantoin contained in the nanoliposome particles produced by ceramide stabilization exhibit more excellent skin improvement activities by a synergistic effect.

As prepared by the stabilization method, a ceramide and a nanoparticle comprising β-sitosterol, resveratrol and allantoin which are bioactive substances show remarkable skin moisturizing effects (Test Examples 3 and 8), anti-oxidation (Test Examples 4 and 5), inflammation relief (Test Example 6), skin barrier enhancement (Test Example 7), and skin itching relief effects (Test Example 9 and Test Example 10), and thus can be useful as a cosmetic material for skin improvement.

The stabilized ceramide as an active ingredient is contained in an amount of 0.0001 to 50.0% (w/w) based on the total weight of the cosmetic composition.

The cosmetic composition is used for skin whitening, moisturizing, skin inflammation relief, skin barrier enhancement, or skin itching relief.

In addition, a solubilizer, a stabilizer, a fragrance, a preservative, a pH adjuster, an antioxidant, and the like may be additionally used within a range that does not impair the object of the present invention.

The cosmetic composition may be prepared in a formulation such as skin lotion, skin toner, pack, nourishing cream, moisture cream, essence, body cream, body lotion, body oil, shampoo, rinse, hair conditioner, hair gel, cleansing foam, cleansing lotion, soap, patch, foundation, lipstick, makeup base, etc.

### EXAMPLE

Hereinafter, the present invention will be described in more detail with reference to the following Examples and Test Examples. However, the following examples are only for illustrating the present invention, and it will be apparent to those skilled in the art to which the present invention pertains that the present invention is not limited by the following examples, and may be changed to other substituted and equivalent embodiments without departing from the technical spirit of the present invention.

### Example 1: Preparation of Nanoparticles Containing Ceramide and a Bioactive Substance

### Preparation of High-Purity Freeze-Dried Powder

In order to remove foreign substances, 10 g of ceramide NP, 1 g of resveratrol, and 0.01 g of β-sitosterol were added and mixed to 100 g of dipropylene glycol heated to a temperature of 45° C. or higher and 900 g of ethanol, and then stirred the same. The solution was allowed to stand at room temperature for 24 hours, filtered to remove precipitates, and then centrifuged to recover the supernatant. The recovered supernatant was concentrated at 45° C. to remove ethanol. It was rapidly frozen at −60° C. or less for 3 hours using Deep Freezer. The uniformly frozen powder is dried in a freeze dryer at 20° C. or less in a vacuum for 4 days (96 hours) to form powder. At this time, the vacuum state means the pressure state of the freeze dryer, and the freezing and drying time may vary depending on the volume of the solution.

### Preparation of Stabilized Ceramide and Bioactive Substance

0.1 g of the freeze-dried powder containing ceramide, resveratrol, and β-sitosterol was dissolved in 10 g of dipropylene glycol, and 2.0 g of phosphatidyl choline, a vegetable phospholipid, was mixed and stirred to prepare an oil phase.

To 80 g of purified water, 8.5 g of dipropylene glycol, 1.5 g of Polyglyceryl-10 Laurate as a solubilizer, and 0.001 g of allantoin were mixed and stirred to prepare an water phase.

The prepared water phase and the oil phase were passed three times through a 2 um flow channel of microfluidic chips incubated at 15° C. or less using a weight ratio of 1:9 to prepare self-assembled liposomes.

Then, the nanoparticle capsule was homogenized at 300 bar using a microfluidizer to prepare a nanoparticle capsule having a uniform size.

### Comparative Example 1: Preparation of a Ceramide-Containing Nano Capsule

0.1 g of ceramide was mixed with 10 g of warmed dipropylene glycol to prepare an oil phase. A nano capsule containing ceramide was obtained in the same manner as in Example 1.

### Comparative Example 2: Preparation of a Mixture of Ceramide and Bioactive Substance

0.1 g of ceramide and 0.01 g of each of the physiologically active substances β-sitosterol, resveratrol, and allantoin were mixed with 20 g of warmed dipropylene glycol and 80 g of purified water to prepare a mixture.

### Test Example 1: Particle Size Analysis of Nanoparticles

The particle size of the nanoparticles prepared in Example 1 was analyzed with a particle size analyzer.

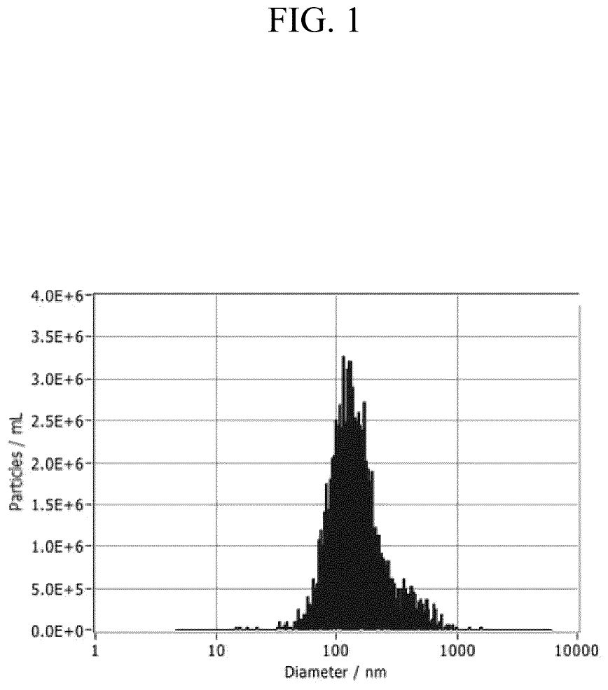

FIG. 1 is a graph showing the analysis result of the particles according to Example 1, and as a result of the analysis, the average size of the particles was confirmed to be 134.2 nm.

### Test Example 2: Evaluation of Cytotoxicity

MTT assay evaluation was performed to confirm the cytotoxicity of the ceramide and bioactive material-containing nanoparticle capsule. Human keratinocytes (HaCaT) were seeded at a concentration of 1×10⁵ cells/mL on a 96 well plate and cultured at 37° C. for 18 hours under 5% CO₂. After the culture, the medium was removed and washed with PBS buffer, and then a new medium and the samples corresponding to Example 1 and Comparative Examples 1 and 2 were administered at different concentrations, and cultured again for 24 hours. In order to measure the cell viability, MTT solution (5 mg/mL) was added thereto, formazan formed for 4 hours was dissolved in Dimethyl sulfoxide (DMSO), and the absorbance was measured at 570 nm using a ELISA reader.

The results are shown in Table 1 below.

TABLE 1

| Cell viability | | Comparative | Comparative |
|---|---|---|---|
| (% of control) | Example 1 | Example 1 | Example 2 |
| Control | 100 | 100 | 100 |
| 0.1% | 104.00 | 105.50 | 100.20 |
| 1% | 103.40 | 103.70 | 98.62 |
| 5% | 100.10 | 100.25 | 94.51 |
| 10% | 95.35 | 93.24 | 88.50 |

As confirmed in Table 1, cytotoxicity was not exhibited in all of the samples of Example 1 and Comparative Examples 1 and 2.

### Test Example 3: Evaluation of Moisturizing Efficacy (AQP3 Expression)

In order to confirm the moisturizing efficacy of ceramide and bioactive substance-containing nanoparticle capsule, the effect on AQP3 expression was evaluated. After inoculating Human dermal fibroblast (HDFa) cell lines, they were cultured at 37° C. for 24 hours under 5% CO₂ in Fibroblast Basal Medium (Medium 106) containing 100 IU/mL penicillin and 100 μg/mL streptomycin. The medium of each well was removed and replaced with fresh serum-free medium. Each well was subjected to pretreatment for 4 hours by administering the samples corresponding to Example 1 and Comparative Examples 1 and 2 at each concentration. 15 mJ/cm² UVB was irradiated to each well plate by a UVB irradiation device (vilber loumet, France). The sample was treated with the diluted serum-free medium, and then further cultured for 24 hours. At this time, a negative control was treated with PBS, and a positive control was treated with Hyaluronic acid at 0.01%. After the cell culture was completed, the cells were lysed using Ribo Ex™ Total RNA Isolation Solution (GeneAll Biotechnology, Korea) and scraper, and then 0.2 mL chloroform (Sigma-Aldrich, USA) was added thereto to perform centrifugation (12,000 rpm, 4° C., 30 minutes). The supernatant containing RNA was separated, and isopropanol (Merck-Millipore, Germany) was added in the same amount as the supernatant, followed by centrifugation (12,000 rpm, 4° C., 30 minutes) after inverting. RNA was precipitated to remove the supernatant except for the precipitate, and 70% of ethanol (Merck-Millipore, Germany) was added thereto to perform centrifugation (12,000 rpm, 4° C., 10 minutes), and washed. Ethanol was removed, dried at room temperature, and dissolved in Nuclease-Free Water (Affymetrix, USA) to extract total RNA. After measuring the purity and concentration of RNA at a wavelength of A260/A280 using MaestroNano® Micro-volume Spectrophotometer (MN-913, Maestrogen, USA), it was confirmed that the ratio of 260 nm and 280 nm was in the range of 2.0 to 2.2. cDNA was synthesized by preparing 1 μg RNA, Oligo dT (Bionics, Korea), dNTP (Takara, Korea), and nuclease free water in PCR tube with total 13 μL and then reacting them at 65° C. for 5 minutes, and then reacting them at 37° C. for 50 minutes using M-MLV Reverse Transcriptase (Invitrogen, Thermo Fisher Scientific, Waltham, Massachusetts, USA). qRT-PCR was performed to quantitatively analyze the gene expression pattern occurring in each cell by the sample. qRT-PCR was performed by mixing primer, cDNA, 2X SYBR Green PCR Master Mix (Applied Biosystems, USA), and HPLC (J. T Baker, USA) in a PCR tube to a total 20 μL, and the reaction was carried out using the StepOnePlus Real-Time PCR System (Applied Biosystems, USA).

The PCR primer sequence of the AQP3 gene is shown in Table 2 below, and the results of evaluating the moisturizing efficacy of the ceramide prepared according to Example 1 and the nanoparticle capsule containing the water-soluble bioactive material at the expression rate of AQP3 mRNA are shown in Table 3 below.

TABLE 2

| Gene | Forward primer (5′→3′) | Reverse primer (5′→3′) |
|---|---|---|
| AQP3 | 5′-CCT TTG GCT TTG CTG CAC | 5′-ACG GGG TTG TTG TAA GGG |
| | TC-3′ | TCA-3′ |
| GAPDH | 5′-GTC TCC TCT GAC TTC AAC | 5′-ACC ACC CTG TTG CTG TAG |
| | AGC G-3′ | CCA A-3′ |

TABLE 3

| Relative AQP3 | Hyaluronic | | | |
|---|---|---|---|---|
| expression | acid | | Comparative | Comparative |
| (% of control) | (0.01%) | Example 1 | Example 1 | Example 2 |
| 0.1% | 149.52 | 105.25 | 100.05 | 103.53 |
| 1% | | 110.72 | 105.42 | 111.54 |
| 5% | | 138.30 | 112.20 | 114.73 |
| 10% | | 155.72 | 125.30 | 122.60 |

As a result of the test, it was confirmed that when the sample of Example 1 was treated, AQP3 mRNA, which is a moisturizing factor involved in cell moisture migration and plays an important role in skin moisturizing, was increased in a concentration-dependent manner, and the expression rate was much higher in the treatment concentration range of Example 1 compared to the samples corresponding to Comparative Examples 1 and 2. At this time, when 0.01% of Hyaluronic acid, which is a positive control, was treated, the expression rate was 149.52%.

### Test Example 4: Evaluation of Anti-Oxidation Efficacy

In order to confirm the anti-oxidation efficacy of ceramide and bioactive material-containing nanoparticle capsules, a DPPH radical scavenging activity test was conducted. The DPPH radical scavenging activity was measured by the reducing power of the sample relative to the DPPH radical. A 0.4 mM solution was prepared by dissolving DPPH (1,1-Diphenyl-2-picrylhydrazyl) reagent in ethanol. Then 100 μL of the solution of each sample corresponding to Example 1, Comparative Examples 1, and 2, or positive control BHT (Butylated hydroxyl toluene or 2,6-Di-tert-butyl-p-cresol) was dissolved in ethanol, was added to 100 μL of the 0.4 mM DPPH solution in a 96 well plate and mixed. At this time, ethanol was treated as a negative control, and after mixing, the mixture reacted at 37° C. for 30 minutes. Absorbance was measured at 517 nm using ELISA reader, and the DPPH radical scavenging ability (%) of the sample or BHT as a positive control was calculated using the following formula.

DPPH radical scavenging ability (%)={1−(absorbance of positive control or sample/absorbance of control)}×100

Table 4 shows the results of evaluating the anti-oxidation effect of each sample by DPPH radical scavenging ability.

TABLE 4

| | DPPH radical scavenging ability (%) | | |
|---|---|---|---|
| Concentration | | Comparative | Comparative |
| (%) | Example 1 | Example 1 | Example 2 |
| 0.1 | 22.28 | 9.25 | 12.00 |
| 1.0 | 29.40 | 10.40 | 15.70 |
| 5.0 | 38.27 | 12.80 | 22.17 |
| 10. | 40.97 | 15.25 | 28.51 |
| BHT | 62.08 | | |
| (0.01%) | | | |

As a result of the test, it was confirmed that when the samples corresponding to Example 1 and Comparative Examples 1 and 2 were treated, the DPPH radical scavenging activity was increased in a concentration-dependent manner, and when the nanoparticles of Example 1 were treated, the highest activity was exhibited.

### Test Example 5: Anti-Oxidation Activity Stability Test

The anti-oxidation activity retention rates of the samples prepared in Example 1 and Comparative Examples were evaluated. The anti-oxidation activity was tested in the same manner as in Test Example 4, and the anti-oxidation retention rates of Examples and Comparative Examples 1 and 2 were compared based on a reference concentration of 5.0%. The anti-oxidation activity was evaluated for each week at a high temperature (45° C.) based on 100% of the anti-oxidation results measured at week 0.

The results are shown in Table 5 below.

TABLE 5

| | | Comparative | Comparative |
|---|---|---|---|
| Weeks | Example 1 | Example 1 | Example 2 |
| Week 0 | 100 | 100 | 100 |
| Week 1 | 91 | 30 | 82 |
| Week 2 | 82 | 25 | 62 |
| Week 4 | 80 | 13 | 35 |

As shown in Table 5, it can be confirmed that when ceramide is stabilized into the nanoliposome, the anti-oxidation activity retention ability in the sample of Example 1 prepared by using β-sitosterol, resveratrol, and allantoin, which are bioactive substances, is far superior to that in the samples of Comparative Examples.

### Test Example 6: Evaluation of Anti-Inflammatory Efficacy

In order to confirm the anti-inflammatory effect, an evaluation of a nitric oxide (NO) production inhibition rate was conducted. Raw264.7 macrophages were dispensed into 96 well plate at 8×10⁴ cells/well and cultured for 24 hours, and then the samples of Example 1 and Comparative Examples 1 and 2 were treated for 1 hour. After treatment, LPS was treated at a concentration of 100 ng/ml and cultured for 24 hours to induce inflammation. Thereafter, 100 μL of the cell culture supernatant and Griess reagent were mixed in the same amount and reacted in a dark place at room temperature for 10 minutes, and then absorbance was measured at 540 nm using ELISA reader. Table 6 is a table showing the results of evaluating the anti-inflammatory efficacy as a rate of inhibiting the production of nitric oxide (NO). As a result of the test, it was confirmed that the amount of NO production decreased in a concentration-dependent manner in all samples, and the NO production inhibitory activity was the best in the sample of Example 1.

TABLE 6

| | NO Production (% of control) | | |
|---|---|---|---|
| Concentration | | Comparative | Comparative |
| (%) | Example 1 | Example 1 | Example 2 |
| Control | 100 | 100 | 100 |
| 0.1 | 92.06 | 98.57 | 90.71 |
| 1.0 | 88.62 | 95.35 | 88.05 |
| 5.0 | 70.18 | 92.70 | 83.62 |
| 10.0 | 59.80 | 87.43 | 79.80 |

### Test Example 7: Evaluation of Skin Barrier Enhancement Efficacy (Filaggrin Expression)

The skin barrier protein is an important element that forms the skin structure and plays an important role in blocking the inflow of external antigens into the body. Types of skin barrier protein include filaggrin, involucrin, and loricrin, and among them, filaggrin plays an important role in skin barrier by aggregating keratin filaments in keratinocytes. Therefore, in order to confirm the skin barrier enhancement efficacy of the nanoparticle capsule of Example 1 containing ceramide and a bioactive substance, an filaggrin gene expression test was conducted compared to Comparative Examples 1 and 2.

After inoculating Human epidermal keratinocyte (HEKa) cell lines, they were cultured at 37° C. for 24 hours under 5% CO₂ in Dulbecco Modified Eagle Medium (DMEM) containing 100 IU/mL penicillin and 100 μg/mL streptomycin. After the culture, the medium was discarded, and the samples of Example 1 and Comparative Examples 1 and 2 were treated and cultured for 48 hours. Then, RNA was extracted from the cultured cells using TransZol reagent, and then RT-PCR (real-time gene polymerase chain reaction) was performed, and the product produced by PCR was electrophoresed in 1% agarose gel and confirmed by Gel Documentation system. At this time, a negative control was treated with PBS, and a positive control was treated with CaCl₂ 20 mM.

TABLE 7

| | Relative FLG expression (% of control) | | |
|---|---|---|---|
| Concentration | | Comparative | Comparative |
| (%) | Example 1 | Example 1 | Example 2 |
| Control | 100 | 100 | 100 |
| 0.1 | 103.35 | 102.18 | 102.25 |
| 1.0 | 121.20 | 116.30 | 101.25 |
| 5.0 | 129.50 | 117.75 | 105.00 |
| 10.0 | 140.25 | 119.42 | 109.67 |

As can be seen in Table 7, when the nanoparticle capsule of Example 1 was treated, the expression of filaggrin was increased in a concentration-dependent manner, and the rate of increase was much higher than when the samples of Comparative Example were treated.

### Example 2: Preparation of a Lotion

A lotion containing the nanoparticle capsule of Example 1 was prepared with the composition of the following Table 8 by a conventional method.

TABLE 8

| Component (wt %) | Content (wt %) |
|---|---|
| Nanoparticles containing ceramide | 3.00 |
| and a bioactive substance (Example 1) | |
| Glycerin | 10.00 |
| Butylene glycol | 5.00 |
| Glyceryl oleate | 1.80 |
| Caprylic/capric triglyceride | 5.00 |
| Cetyl ethyl hexanoate | 1.00 |
| Beads wax | 0.50 |
| Squalane | 0.20 |
| 1,2-hexanediol | 2.00 |
| Demethicone | 0.50 |
| Cyclopentasillon/cyclohexyloxane | 2.00 |
| Cetiaryl alcohol | 1.00 |
| Mineral oil | 2.50 |
| Disodium EDTA | 0.02 |
| BHBT | 0.05 |
| Tocopheryl acetate | 0.30 |
| Panthenol | 0.20 |
| Ethylhexylmethoxycinnamate | 0.20 |
| Fragrances | 0.01 |
| Purified water | Residual |
| | amount |

### Comparative Examples 3 to 4: Preparation of a Lotion

In Example 2, a lotion was prepared in the same manner as in Example 1, except that Comparative Example 1 was added instead of the nanoparticles of Example 1 (Comparative Example 3). In addition, a lotion containing 3.00% of hyaluronic acid, which is known to have a moisturizing effect, was prepared as Comparative Example 4.

### Test Example 8: Effect of Improving Skin Moisturizing Ability

To evaluate the effect of improving skin moisturizing, the following experiment was conducted. A total of 60 participants aged 20 to 40 years without skin diseases were divided into 3 groups of 20 people each. Each group applied the lotion from Example 2, Comparative Example 3, and the lotion containing hyaluronic acid, a substance known to have a moisturizing effect (Comparative Example 4), to the face and forearms twice daily for one month.

The skin conductivity was measured using corneometer (CM820 courage Kazaka electronic GmbH) under constant temperature and humidity conditions (24° C., humidity 40%) before starting the application in advance to use it as a basic value, and the skin conductivity was measured after 1 week, 2 weeks, and 4 weeks to evaluate the increase rate. The results are shown in Table 9 below.

TABLE 9

| Sample name | After 1 week | After 2 weeks | After 4 weeks |
|---|---|---|---|
| Example 2 | 45 | 48 | 49 |
| Comparative | 22 | 27 | 35 |
| Example 3 | | | |
| Comparative | 42 | 45 | 46 |
| example 4 | | | |

As shown in the results of Table 9, the lotion of the formulation of Example 2 containing the ceramide and the bioactive material-containing nanoparticle capsule of the present invention had a very excellent skin conductivity increase rate compared to Comparative Example 3. In addition, compared to Comparative Example 4, which is a cosmetic material containing hyaluronic acid known to have a moisturizing effect, it showed a similar increase in skin conductivity.

In general, since the skin conductivity is proportional to the skin moisture content, it can be confirmed from the above results that the cosmetic material containing the present invention also maintain a higher skin moisture content than the cosmetic material containing purified water.

### Test Example 9: Effect of Skin Itching Relief

The skin itching Relief improvement effects of Example 2 and Comparative Examples 3 and 4 were comparatively measured in 30 adults (average age 42.0 years) who reported skin itching. The experiment was conducted in a temperature- and humidity-controlled room at 22° C. with 45% relative humidity.

The present invention evaluated the degree of skin irritation using a 10-point scale before using each formulation for each group (1 to 3 points: unconscious scratches (life, no life disturbance), 4 to 6 points: Feeling of insomnia (not a full-time day), 7 to 9 points: Feeling of insomnia during most of the time, 10 point: Severe difficulty in life and sleep is caused by itching. Example 2 and Comparative Examples 3 and 4 were distributed, and used twice a day in the morning and in the evening for 4 weeks. After 2 weeks and 4 weeks, the degree of skin itching was evaluated again by subjective evaluation to evaluate the degree of improvement of the itching. The amount of change compared to the initial value is shown in Table 10.

TABLE 10

| Sample name | Before use | After 2 weeks | After 4 weeks |
|---|---|---|---|
| Example 2 | 4.5 | 2.5 | 2.0 |
| Comparative Example | 5.0 | 4.5 | 4.5 |
| 3 | | | |
| Comparative example | 4.0 | 3.5 | 2.5 |
| 4 | | | |

As shown in the results of Table 10, it was confirmed that the lotion of the formulation of Example 2 containing the ceramide and the bioactive substance of the present invention had an excellent effect of improving skin irritation, compared to Comparative Example 3.

### Test Example 10: Evaluation of Skin Irritation

A degree of skin irritation was evaluated, and an experiment was performed as follows to determine whether a safe formulation is provided in which there is no skin irritation and no itching occurs.

The experiment was conducted on 15 people in their 20s and 40s who had no skin disease. The product was dropped in the patch holding chamber at 20 ul each time, volatile substances were volatilized for 1 hour, and then a patch was attached to a subject, etc., and after 24 hours, the patch was removed and readout was performed after 30 minutes. The results are shown in Table 11.

TABLE 11

| Sample name | Skin irritation % |
|---|---|
| Example 1 | 0.00 |
| Comparative Example 1 | 0.00 |
| Comparative Example 2 | 0.20 |
| Example 2 | 0.00 |
| Comparative Example 3 | 0.00 |
| Comparative example 4 | 0.00 |

As shown in the results of Table 11, it was confirmed that the stimulation was not high in Examples 1 and 2 and Comparative Examples 1, 3, and 4, except for Comparative Example 2.

Ceramide formulation activity stability is improved by homogenizing ceramide with β-sitosterol, resveratrol, and allantoin which are other physiologically active materials using a microfluidic chip and a high pressure homogenizer. Ceramide prepared by the stabilizing method of the present invention and nanoparticles comprising β-sitosterol, resveratrol, and allantoin which are physiologically active materials have high skin absorption rate to stay on the skin for a long time and stably absorb the active materials, thereby exhibiting excellent moisturizing, anti-oxidation, skin inflammation relief, skin barrier enhancement, and skin itching relief, and can be useful as a cosmetic material for skin improvement.

What is claimed is:

1. A method for stabilizing ceramide, comprising:
(A) preparing freeze-dried powder containing ceramide, resveratrol, and β-sitosterol;
(B) b1) dissolving the freeze-dried powder in dipropylene glycol, followed by mixing an emulsion polymer to prepare an oil phase;
(b2) mixing purified water, the dipropylene glycol, a water-soluble solubilizer, and allantoin to prepare a water phase;
(C) self-assembling the oil phase and the water phase by passing through a microfluidic chip; and
(D) forming nanoparticles using a microfluidizer.

2. The method for stabilizing the ceramide of claim 1, wherein the step (A) comprises steps of:
a1) adding ceramide, resveratrol, and β-sitosterol to warm dipropylene glycol and ethanol, followed by mixing, and dissolving;
a2) subjecting the mixture to stand at room temperature followed by filtration to remove a precipitate; and
a3) concentrating the mixture at 40 to 50° C., and freeze-drying the concentrate to prepare freeze-dried powder containing ceramide, resveratrol, and β-sitosterol.

3. The method for stabilizing ceramide of claim 1,
wherein the emulsion polymer is at least one selected from a group consisting of phosphatidyl choline, Poly-ε-caprolactone (PCL), polyglycolic acid (PGA), poly-L-lactide (PLLA), sphingomyelin, phosphatidyl serine, phosphatidyl inositol, phosphatidyl ethanolamine, and lecithin.

4. The method for stabilizing ceramide of claim 1,
wherein the ceramide is at least one selected from a group consisting of ceramide NP, ceramide EOP, ceramide NS, ceramide AS, ceramide EOS, and ceramide NDS.

5. The method for stabilizing ceramide of claim 2,
wherein in step a1), the ceramide, the resveratrol and the β-sitosterol are added and mixed at a weight ratio of 10:0.5 to 1.5:0.005 to 0.02, respectively.

6. The method for stabilizing ceramide of claim 1,
wherein in step b1), the oil phase is formed by mixing 0.5 to 1 wt % of the freeze-dried powder, 80 to 85 wt % of the dipropylene glycol and 14 to 18 wt % of the emulsion polymer.

7. The method for stabilizing ceramide of claim 1,
wherein in step b2), the water phase is formed by mixing 88 to 90 wt % of purified water, 8 to 10 wt % of dipropylene glycol, 1 to 2 wt % of a water-soluble solubilizer and 0.0005 to 0.002 wt % of allantoin.

8. The method for stabilizing ceramide of claim 1,
wherein in step (C), the oil phase and water phase are used in a ratio of 1:7 to 1:12 and passed through a microfluidic chip in which a microinjection port of 1 um to 3 um is formed, at a temperature of 15° C. or lower, preferably 10 to 15° C., at least once, and preferably 1 to 5 times, to prepare self-assembled liposomes.

9. The method for stabilizing ceramide of claim 1,
wherein in step (D), the nanoparticles have a size of 50 to 300 nm.

10. A cosmetic composition comprising 0.0001 to 50.0 wt % of ceramide stabilized by the method according to claim 1, based on total weight of the cosmetic composition.

11. The cosmetic composition of claim 10,
wherein the cosmetic composition is for skin moisturizing.

12. The cosmetic composition of claim 10,
wherein the cosmetic composition is for anti-oxidation.

13. The cosmetic composition of claim 10,
wherein the cosmetic composition is for skin inflammation relief.

14. The cosmetic composition of claim 10,
wherein the cosmetic composition is for skin barrier enhancement.

15. The cosmetic composition of claim 10,
wherein the cosmetic composition is for skin itching relief.
